(19) United States
(12) Patent Application Publication
(10) Pub. No.: US 2025/0099357 A1
(43) Pub. Date: Mar. 27, 2025

(54) ANTIMICROBIAL SYSTEM WITH BETA CARBOLINE ALKALOID AND PHENOLIC ACID AND COMPOSITIONS COMPRISING THEM

(71) Applicant: Conopco, Inc., d/b/a UNILEVER, Englewood Cliffs, NJ (US)

(72) Inventors: Jose Guillermo Rosa, Cheshire, CT (US); Matthew Joseph RIENZO, Fairfield, CT (US); Deidre Lee MITCHEL, Oxford, CT (US); Bijan HARICHIAN (Deceased)

(73) Assignee: Conopco, Inc., d/b/a UNILEVER, Englewood Cliffs, NJ (US)

(21) Appl. No.: 18/722,521

(22) PCT Filed: Dec. 19, 2022
(86) PCT No.: PCT/EP2022/086552

§ 371 Date: Jun. 20, 2024

(30) Foreign Application Priority Data
Dec. 27, 2021 (EP) 21217788.5

(51) Int. Cl. A61K 8/49; A61K 8/365; A61Q 17/00

(52) U.S. Cl. CPC A61K 8/4926; A61K 8/365; A61Q 17/005

(57) ABSTRACT

Antimicrobial systems with naturally derived ingredients and compositions comprising them are described. The systems have a first ingredient comprising a beta carboline alkaloid and a second natural ingredient that includes a phenolic acid where the systems and compositions provide superior antimicrobial benefits.

### FIELD OF THE INVENTION

The present invention is directed to an antimicrobial system comprising naturally derived components. More particularly, the invention relates to a beta carboline alkaloid, and phenolic acid and/or salts thereof comprising antimicrobial systems that can be included in end use formulations, including cosmetic, pharmaceutical, home care and wash compositions. The components of the antimicrobial system of the present invention are found naturally in fruits and plants, and unexpectedly, deliver superior and synergistic antimicrobial benefits when added to such end use compositions. The end use compositions comprising the antimicrobial system of the present invention can be formulated substantially free of parabens, formaldehyde donors, triclosan as well as other traditional antimicrobial components including sulphated and halogenated antimicrobial ingredients.

### BACKGROUND OF THE INVENTION

DMDM hydantoin, parabens, methylisothiazolinone as well as methylchloroisothiazolinone are commonly used preservatives (i.e., antimicrobial components) found in consumer products. Such components have been used for years and are known to work well at maintaining the integrity and stability of certain end use compositions. Notwithstanding, there is a desire to develop antimicrobial systems that include naturally occurring components suitable to work well across a full range of consumer products. Naturally occurring microbial systems should be effective at preserving products, not be skin sensitizing and not negatively impact the sensorial characteristics of consumer products, especially those that are topically applied.

In addition to delivering superior antimicrobial benefits, the systems should not be harmful to the environment and gentle enough for use on the most fragile consumer, babies.

There is increasing interests to develop antimicrobial systems that are effective, not skin sensitizing and environmentally sustainable. This invention, therefore, is directed to an antimicrobial system with naturally occurring ingredients as well as compositions that comprise the same. The antimicrobial system comprises a beta carboline alkaloid, and phenolic acid and/or salts thereof. Such a system unexpectedly delivers superior and synergistic antimicrobial benefits to end use compositions they are used in.

### Additional Information

Efforts have been disclosed for making preservative systems. In U.S. Published Patent Application 2012/0190744A1, preservative systems for cosmetic formulations are described.

Still other efforts have been disclosed for making preservative systems. In U.S. Pat. No. 7,754,774B2, antiseptic disinfectants with 1,2-octane diol are described

Even other efforts have been disclosed for making preservative systems. In U.S. Pat. No. 7,582,681B2, antimicrobial active compounds with 1,2-alkane diols are described.

Additional efforts have been disclosed for modulating inflammatory responses. In World Application, 2021/202982 A1, described is a composition comprising an aqueous or solid fraction of a mushroom mycelium and/or fruit body mixture to, for example, treat prophylaxis of, or ameliorating symptoms of one or more adverse reactions triggered by an infectious disease.

None of the additional information above describes an antimicrobial system and end use composition with such a system as described and claimed herein.

### SUMMARY OF THE INVENTION

In a first aspect, the present invention is directed to an antimicrobial system comprising:
(a) a first component comprising a beta carboline alkaloid; and
(b) a second component comprising a phenolic acid, a phenolic acid salt or both.

In a second aspect, the present invention is directed to an end use composition comprising:
(a) a first component comprising a beta carboline alkaloid; and
(b) a second component comprising a phenolic acid, a phenolic acid salt, or both.

In a third aspect, the present invention is directed to a use or method for preserving a composition with an antimicrobial system having a first component comprising a beta carboline alkaloid, and a second component comprising a phenolic acid, a phenolic acid salt, or both.

In a fourth aspect, the present invention is directed to a use or method for providing an antimicrobial benefit to a consumer by contacting skin with a composition having a first component comprising a beta carboline alkaloid, and a second component comprising a phenolic acid, a phenolic acid salt, or both.

In a fifth aspect, the present invention is directed to a use or method for providing an antimicrobial benefit to an object, like a table surface, by contacting the object with a composition having a first component comprising a beta carboline alkaloid, and a second component comprising a phenolic acid, a phenolic acid salt, or both.

All other aspects of the present invention will more readily become apparent from the description and examples which follow.

Antimicrobial system, as used herein, means at least a two-component and natural system suitable to show a synergistic antimicrobial reduction, including an antimicrobial benefit as seen with preservatives in a composition and as topically applied to a surface, including skin or a home care surface like a table surface. The antimicrobials surprisingly reduced synergistically with the system of the present invention include Enterobacter gergoviae ATCC 33028 of the family Pluralibacter gergoviae, a Gram-negative, rod-shaped bacterium that often displays resistance to parabens, or such bacterium and Aspergillus brasiliensis ATCC 16404, a fungus of the genus Aspergillus. The at least two component system means comprising at least one component that is a beta carboline alkaloid and at least one component that is a phenolic acid (i.e., can be sold as a separate additive composition for adding to an end use composition or added separately along with the ingredients to make the desired end use composition). Naturally derived means not requiring synthetic manufacturing and suitable to be recovered from (at least in one form) a fruit, plant and/or root. Naturally derived, therefore, includes any analogues/derivatives of the components that are ready to use as extracted or isolated from a natural resource. In the case of phenolic acids, for example, naturally derived includes a component like syringic acid that is naturally recovered from a fruit like sea buckthorn berries and any ether or ester variations made therefrom. Salt as used herein in relation to phenolic acid means, for example, having a sodium, potassium, calcium, magnesium and/or ammonium cation.

Skin, as used herein, includes skin on the feet, face, neck, chest, arms (including under arms), hands, legs, buttocks, back and scalp (including hair). The end use composition described herein (i.e., a composition ready to use or apply) includes creams, lotions, serums, gels, balms, deodorants and antiperspirants, oral care compositions, shampoos, conditioners, bars and liquid wash products as well as home care compositions like hard surface and window cleaners, toilet bowl cleaners and laundry detergents. In one embodiment, the end use composition of the present invention is a cleaning composition, a wash product or a leave-on product, such as a cream or lotion to be applied to the face, body or hands. In another embodiment, the end use composition is a cosmetic leave-on product suitable to cosmetically improve a skin attribute such as reducing the appearance of wrinkles and/or moisturizing skin. Such a composition can also be one that results in skin having an even colour or tone as well as reduced hyperpigmentation spots caused by environmental factors, the aging process, or both.

Unless explicitly stated otherwise, all ranges described herein are meant to include all ranges subsumed therein. The term comprises is meant to encompass the terms consisting essentially of and consisting of. For the avoidance of doubt, a composition comprising a preservative portion with syringic acid and harmol is meant to include a composition consisting essentially of the same and a composition consisting of the same. As to the percentages used herein, the same are meant to be by weight unless noted otherwise. Except in the operating and comparative examples, or where otherwise explicitly indicated, all numbers used in this description indicating amounts, or ratios of materials and/or use thereof are to be understood as modified by the word “about”. Substantially free of, as used herein, means less than 1.5% or less than 0.35% by weight of the total weight of the end use composition.

### DETAILED DESCRIPTION OF THE INVENTION

There is no limitation with respect to how the component which comprises a beta carboline alkaloid used in the present invention is naturally sourced except that obtaining of the same is preferably achieved in a most sustainable manner. Often, a green extraction process using ethyl acetate and sodium bicarbonate is used; especially, in lieu of common extraction processes that use chloroform. The natural sources of the beta carboline alkaloid used in the present invention are, for example, fruits, like sea buckthorn berries, and plants, like the roots of the Peganum Harmala plant. The alkylation of the naturally sourced component is typically achieved by reacting the alkaloid with an olefin in the presence of an acid catalyst, or alternatively, by reacting the alkaloid with an alkyl group containing a leaving group (i.e., nucleofuge) in the presence of a base.

The illustrative beta carboline alkaloids suitable for use in the invention are represented as:

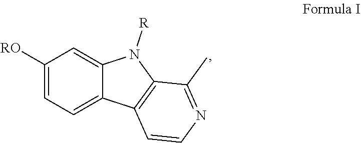

where each R is independently hydrogen or a C₁-C₄ alkyl.

In an embodiment of the invention, —OR is a hydroxy group and —NR is —NH and the beta carboline alkaloid is 1-Methyl-9H-pyrido[3,4-b]indol-7-ol, commonly called Harmol.

As to the components comprising a phenolic acid or salt thereof suitable for use in the present invention, there is no limitation with respect to how such component is naturally sourced except that the recovery of the same is preferably achieved in a most sustainable manner. Natural sources of the components comprising phenolic acid are fruits including olives, dates, pumpkin and grapes, as well as spices, acai palm, honey and red wine. The phenolic acid may be recovered from such sources via art recognized techniques that include extractions via solvents like ethanol, methanol, ethyl acetate or ethyl ether and even water as phenolic acids are slightly soluble in water.

Illustrative examples of the phenolic acid suitable for use in the invention are represented as:

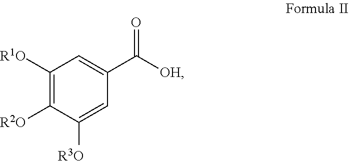

where each of R¹, R² and R³ is independently hydrogen or a C₁-C₄ alkyl.

In an embodiment of the invention, R¹ and R³ are CH₃ and R² is H and the acid is 4-Hydroxy-3,5-dimethoxybenzoic acid, commonly referred to as Syringic acid. In another embodiment, each of R¹, R² and R³ is H and the acid is 3,4,5-trihydroxybenzoic acid, commonly referred to as Gallic acid. In still another embodiment, each of R¹, R² and R³ is CH₃, and the acid is 3,4,5-trimethoxybenzoic acid, commonly referred to as Eudesmic acid. Mixtures of such acids are suitable for use as are salts of the same. As to derivatives and analogues of the same, these can be prepared by subjecting the acid naturally occurring to art recognized alkylation and dealkylation processing steps.

The weight ratio of carboline alkaloid to phenolic acid (and/or salts thereof) used in the present invention is often from 0.75:8 to 0.75:48, and preferably, from 0.85:4 to 1:44, and most preferably, from 0.95:3 to 2:40. In an embodiment of the invention and when it is desirable for the end use compositions to be effective on bacteria, the antimicrobial system preferably comprises from 0.7 to 5%, and preferably, from 0.8 to 4%, and most preferably, from 0.9 to 3% by weight carboline alkaloid based on total weight of carboline alkaloid and phenolic acid in the antimicrobial system. In another embodiment and when it is desirable for the end use compositions to be effective on both bacteria and fungus, the antimicrobial system preferably comprises from 45 to 78%, and preferably, from 50 to 75%, and most preferably, from 58 to 70% by weight carboline alkaloid based on total weight of carboline alkaloid and phenolic acid (and/or salts thereof) in the antimicrobial system.

Typically, the end use compositions made with the antimicrobial system of the present invention will have a total weight of antimicrobial system (i.e., carboline alkaloid and phenolic acid) from 0.001 to 12% based on total weight of the end use composition. In an embodiment of the invention, the composition will have from 0.01 to 10%, and still another embodiment, from 0.1 to 8% by weight antimicrobial system based on total weight of the end use composition. In yet another embodiment, the end use composition will have a total weight of antimicrobial system from 1 to 6% or from 1.25 to 6% or from 1.5 to 5% or from 2 to 4.5% by weight based on total weight of the end use composition.

There is generally no limitation with respect to the type of end use composition that may comprise the antimicrobial system of the present invention. End use compositions that are suitable to use the antimicrobial system of this invention will typically include cosmetically acceptable carrier components. Water is the most preferred carrier. Amounts of water may range from 1 to 96%, and preferably from 5 to 90%, and most preferably, from 35 to 80%, and optimally, from 40 to 75% by weight, based on total weight of the end use composition. Ordinarily the end use compositions of this invention will be water and oil emulsions, most preferably, of the oil-in-water variety. Water-in-oil emulsions, and especially, those generally classified as water-in-oil and high internal phase emulsions are, however, an option. Illustrative examples of the high internal phase emulsions suitable to include the antimicrobial system of this invention are described in commonly owned U.S. Patent Application Publication No. 2008/0311058 and U.S. Pat. No. 8,425,882, the disclosures of which are incorporated herein by reference.

Other cosmetically acceptable carriers suitable for use in this invention may include mineral oils, silicone oils, synthetic or natural esters, and alcohols. Amounts of these materials may range from 0.1 to 50%, and preferably, from 0.1 to 30%, and most preferably, from 1 to 20% by weight of the end use composition, including all ranges subsumed therein. In still another embodiment, such carriers collectively make up from 1 to 12% or from 1 to 8% by weight of the end use composition.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, and preferably, from 4 to 5 silicon atoms.

Linear volatile silicone materials generally have viscosities of less than 5 centistokes at 25° C. while cyclic materials typically have viscosities of less than 10 centistokes (measured with a Brookfield Viscometer, RV No. 3 spindle at 20 RPM, standardized to mineral oil).

Nonvolatile silicone oils useful as carrier material include polyalkyl siloxanes, polyalkylaryl siloxanes and polyether siloxane copolymers. The essentially non-volatile polyalkyl siloxanes useful herein include, for example, polydimethylsiloxanes (like dimethicone) with viscosities of from 5 to 100,000 centistokes at 25° C.

An optional silicone source is a cyclopentasiloxane and dimethiconol solution.

Among suitable esters are:
(1) alkyenyl or alkyl esters of fatty acids having 10 to 20 carbon atoms like isopropyl palmitate, isopropyl isostearate, isononyl isonanonoate, oleyl myristate, isopropyl myristate, oleyl stearate, and oleyl oleate;
(2) ether-esters such as fatty acid esters of ethoxylated fatty alcohols;
(3) polyhydric alcohol esters such as ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid esters, sorbitan fatty acid esters, and polyoxyethylene sorbitan fatty acid esters;
(4) wax esters such as beeswax, spermaceti, myristyl myristate, stearyl stearate; and
(5) sterol esters, of which soya sterol and cholesterol fatty acid esters are examples thereof.

Emulsifiers (or surfactants) may be present in the end use compositions comprising the antimicrobial system of the present invention. Total concentration of the emulsifier may range from 0.1 to 30%, and preferably, from 2 to 20%, and most preferably, from 1 to 8% by weight of the end use composition. The emulsifier may be selected from the group consisting of anionic, nonionic, cationic and amphoteric components. Particularly preferred nonionic components are those with a C₁₀ to C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers.

Suitable anionic emulsifiers that may be used include alkyl ether sulfate and sulfonates, alkyl sulfates and sulfonates, alkylbenzene sulfonates, alkyl and dialkyl sulfosuccinates, C₈-C₂₀ the end use composition. In a preferred embodiment of the invention, no traditional preservative, vicinal diol and/or fragrance component (except for what may be provided in the fragrance used in the end use composition) is used in the end use composition since such compositions comprise the antimicrobial system of the present invention. In another embodiment of the invention, the end use composition has no (0.0% by weight) paraben, formaldehyde donor and/or phenoxyethanol. In yet another embodiment of the invention, the end use composition is substantially free of silicone and/or sulfate comprising components like surfactants or emulsifiers. In even another embodiment, the end use composition of the present invention is silicone free and/or sulfate comprising component free (i.e., 0.0% by weight).

Thickening agents may optionally be included in end use compositions of the present invention. Particularly useful are the polysaccharides. Examples include citrus fibers, starches, natural/synthetic gums and cellulosics. Representative of the starches are chemically modified starches such as sodium hydroxypropyl starch phosphate and aluminium starch octenylsuccinate. Tapioca starch is often preferred. Suitable gums include xanthan, sclerotium, pectin, karaya, Arabic, agar, guar, carrageenan, alginate and combinations thereof. Suitable cellulosics include hydroxypropyl cellulose, hydroxypropyl methylcellulose, ethylcellulose and sodium carboxy methylcellulose. Synthetic polymers are yet another class of effective thickening agent. This category includes crosslinked polyacrylates such as the Carbomers, polyacrylamides such as Sepigel® 305 and taurate copolymers such as Simulgel EG® and Aristoflex® AVC, the copolymers being identified by respective INCI nomenclature as Sodium Acrylate/Sodium Acryloldimethyl Taurate and Acryloyl Dimethyltaurate/Vinyl Pyrrolidone Copolymer. Another preferred synthetic polymer suitable for thickening is an acrylate-based polymer made commercially available by Seppic and sold under the name Simulgel IN100.

Amounts of the thickening, when used, may range from 0.001 to 5%, and preferably, from 0.1 to 2%, and most preferably, from 0.2 to 0.5% by weight of the end use composition.

Fragrances, fixatives and abrasives may optionally be used in the end use compositions that include the antibacterial systems of the present invention. Each of these substances may range from 0.05 to 5%, preferably between 0.1 and 3%, and most preferably, from 0.2 to 1% by weight of the total weight of the end use composition.

Conventional humectants may be employed in the end use compositions of the present invention. These are generally polyhydric alcohol-type materials. Typical polyhydric alcohols include glycerol (i.e., glycerine or glycerin), propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. Most preferred is glycerine, propylene glycol or a mixture thereof. The amount of humectant employed may range anywhere from 0.5 to 20%, preferably between 1 and 15%, and most preferably, from 2 to 10% or even 2 to 4% by weight of the end use composition.

The end use compositions of the present invention may include vitamins. Illustrative vitamins are Vitamin A (retinol) as well as retinol esters like retinol palmitate and retinol propionate, Vitamin B2, Vitamin B3 (niacinamide), Vitamin B6, Vitamin C, Vitamin A, Vitamin D, Vitamin E, Vitamin K, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of vitamins when present may range from 0.001 to 10%, and preferably from 0.01% to 5%, optimally from 0.1 to 2% by weight of the end use composition.

Other optional additives suitable for use in this invention include resorcinols, like thiamidol, 4-ethyl resorcinol, 4-hexyl resorcinol, 4-phenylethyl resorcinol, dimethoxytoluyl propyl resorcinol, 4-cyclopentyl resorcinol, 4-cyclohexylresorcinol, alpha- and/or beta-hydroxyacids, petroselinic acid, conjugated linoleic acid, 12-hydroxystearic acid, mixtures thereof or the like. Still other optional additives like ethanol, quaternary ammonium compounds (like cetrimonium chloride, benzalkonium chloride or the like) and lecithin may also be included. Such additives, when used, collectively make up from 0.001 to 12%, and preferably, from 0.01 to 6%, and most preferably, from 0.1 to 4% by weight of the end use composition. In an embodiment of the invention, these optional additives make up, individually and when used, from 0.01 to 1% by weight of the end use composition.

Desquamation promoters may be present in the end use compositions of the present invention. Illustrative are the alpha-hydroxycarboxylic acids, beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic and its derivatives, lactic and malic acids. Salicylic acid is representative of the beta-hydroxycarboxylic acids. Amounts of these materials when present may range from 0.001 to 15%, and preferably, from 0.01 to 6%, and most preferably, from 0.1 to 3% by weight of the end use composition.

A variety of herbal extracts may optionally be included in the end use compositions of this invention. The extracts may either be water soluble or water-insoluble and carried in a solvent which respectively is hydrophilic or hydrophobic. Water and ethanol are the preferred extract solvents. Illustrative extracts include those removed from green tea, yarrow, chamomile, licorice, aloe vera, grape seed, citrus unshiu, willow bark, sage and rosemary.

Also, optionally suitable for use are materials like chelators (e.g., EDTA), opacifiers (like TiO₂, particle size from 50 to 1200 nm, and preferably, 50 to 350 nm), C₈-22 fatty acid substituted saccharides, lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be optionally included. Amounts of these materials may range from 0.0001 to 8%, and preferably, from 0.001 to 6%, and most preferably, from 0.001 to 3% by weight of the end use composition, if they are used.

Sunscreen actives may also be optionally included in end use compositions of the present invention. Particularly preferred are such materials as ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide, zinc oxide, polyethylene and various other polymers. Amounts of the sunscreen agents when present may generally range from 0.1 to 30%, preferably from 0.5 to 20%, optimally from 0.75 to 10% by weight.

Conventional buffers/pH modifiers may be used. These include commonly employed additives like sodium hydroxide, potassium hydroxide, hydrochloric acid, citric acid, triethanolamine, citrate/citric acid buffers or mixtures thereof. In an embodiment of the invention, the pH of the end use composition is from 4 to 8, and preferably, from 4.25 to 7.75, and most preferably, from 5.6 to 7.5.

In yet another embodiment of the invention and when the end use composition is a conventional cleansing or wash composition, the same often comprises 4 to 45% or from 6 to 28% or from 8 to 22% by weight C₈ to C₂₀, preferably C₁₀ to C₂₀ or C₁₂ to C₁₈ fatty acid or fatty acid soap and less than 5% by weight synthetic surfactant, and preferably, 1 to 3% by weight synthetic surfactant (syndet). In an embodiment of the invention, the cleansing or wash composition comprises from 0 to 5%, and preferably, from 0.01 to 4% by weight 12-hydroxystearic acid (12-HSA) and from 0.01 to 25% by weight glycerol. In still another embodiment, the end use composition of the present invention comprises from 0 to 6% by weight sulfate-based surfactant (e.g., sodium lauryl sulfate), but preferably no (0.0% by weight) sulfate-based surfactant.

In even another embodiment of the invention and when the end use composition is a cleansing or wash composition, the composition can comprise less than 8% by weight fatty acid and fatty acid soap. When the case, syndet makes up from 2 to 30%, and preferably, 3 to 22%, and most preferably, from 4 to 15% by weight of the end use composition having the antimicrobial system of the present invention. The syndets used are typically a glycinate, glutamate, isethionate, taurate, betaine or a mixture thereof.

The viscosity of the end use composition of this invention is typically from 1,000 to 50,000 cps. The wash off composition preferably has a viscosity under 9,000 cps and the leave-on compositions preferably have a viscosity at 6,000 to 15,000 cps. Viscosity may be measured with art recognized instrumentation such as a Brookfield Viscometer RVT, Model D220, using a T-bar spindle D at 5 RPM, 60 seconds at 25° C.

Illustrative end use compositions that may be made to include from 0.001 to 12% by weight of the antimicrobial system of this invention are described below.

Body Lotion for Shower

| Ingredient | Weight % |
|---|---|
| Water | Balance |
| Hydroxypropyl Starch Phosphate | 2.0 |
| Cetyl Phosphate | 1.0 |
| Potassium Hydroxide | 0.5 |
| Hydroxypropyl Bis-Hydroxyethyl Dimonium Chloride | 3.0 |
| Stearamidopropyl PG-Dimonium Chloride Phosphate | 2.0 |
| Shea Butter | 5.0 |
| Coconut Oil | 5.5 |
| Cococaprylate | 2.0 |
| Cetearyl Alcohol | 1.5 |
| Fragrance | 0.4 |

Body Lotion

| Ingredient | Weight % |
|---|---|
| Water | Balance |
| Cetyl Hydroxyethylcelllose | 0.25 |
| Sorbitan Oleate Decylglucoside Crosspolymer | 3.5 |
| Potassium Cetyl Phosphate | 1.0 |
| Glycerin | 2.0 |
| Ethylhexyl Palmitate | 3.0 |
| Isopropyl Palmitate | 6.0 |
| Avocado Oil | 3.0 |
| Cetyl Alcohol | 4.0 |
| Diheptyl Succinate and Capryloyl Glycerin/ | 2.0 |
| Sebacic Acid Copolymer | |
| Fragrance | 0.4 |

Shampoo Composition

| Ingredient | Weight % |
|---|---|
| Water | Balance |
| Cocamidopropyl Betaine | 20.0 |
| Sodium Bis-Hydroxyethylglycinate Coco- | 6.0 |
| Glucoside Crosspolymer | |
| Sodium Methyl Cocoyl Taurate | 2.0 |
| Sodium Stearoyl Lactylate | 10.0 |
| Fragrance | 0.8 |
| pH Adjuster | 0.4 |
| Traditional Preservative* | 0.6 |

*Sodium Benzoate or Phenoxyethanol

Body Wash Composition

| Ingredient | Weight % |
|---|---|
| Water | Balance |
| Surfactants | 8-20 |
| Opacifier | 0-2 |
| Humectant | 3-8 |
| Thickener | 0.3-1.5 |
| Chelator | 0.1-1 |
| Fragrance | 0.5-1.2 |

When preparing such compositions, stirring is used at atmospheric pressure with moderate heat (50 to 70° C.) until homogeneous compositions are obtained. Homogenization is preferably included. The antimicrobial system may be added as combined components or as individual components to the desired end use composition where water is added to balance.

A wide variety of packaging can be employed to store and deliver the end use composition comprising the antimicrobial system of this invention. Packaging is often dependent upon the type of product and its use. For instance, leave-on skin lotions and creams, shampoos, conditioners and shower gels generally employ plastic containers with an opening at a dispensing end covered by a closure. Metallic can pressurized by a propellant and having a spray nozzle serve as packaging for mousses and other personal care products like antiperspirants. Post-consumer resins are often preferred.

It is within the scope of the invention to also include the antimicrobial system in concentrates (i.e., precursor end use compositions) where the consumer obtains such concentrate and adds water to the same to generate the desired end use composition.

The following example is provided to facilitate an understanding of the present invention. The example is not intended to limit the scope of the claims.

The surprising synergistic benefits obtained when using the microbial system of the present invention were confirmed by performing studies similar to the one described below.

Naturally derived antimicrobial systems were made, as shown below by combining the beta carboline alkaloid and phenolic acid. Two pools were set up and each pool contained 1 organism. The strains involved were Enterobacter gergoviae ATCC 33028 of the family Pluralibacter gergoviae, a Gram-negative and Aspergillus brasiliensis ATCC 16404. The FIC test range was 16 times greater than the projected MIC for each sample. The first and second natural components (Components A and B) were combined to make a mixture of natural components and antimicrobial system to demonstrate the unexpected and superior results obtained.

The differing behaviors of inhibitory antimicrobials in isolation and mixtures have been widely explored using the concept of the Fractional Concentration and Fractional Inhibitory Concentration (FIC). See for instance JRW Lambert and R Lambert, J. Appl. Microbiol 95, 734 (2003); T. Jadavji, CG Prober and R Cheung, Antibacterial Agents and Chemotherapy 26, 91 (1984), and WO 2004/006876. MIC is the minimum inhibitory concentration a component prevents visible growth of a microbe. The FC and FIC parameters can be defined as follows:

$$FC ( Component A ) = Concentration of Component A tested in the mixture / MIC ( Component A tested as a single active )$$

$$FIC ( component a ) = MIC ( component a tested in the mixture ) / MIC ( component a tested as a single active )$$

The interactions between antimicrobials can be additive, synergistic or possibly antagonistic depending on whether the efficacy of the combination is equivalent to, greater than or less than that obtained for the same total concentration of the individual components when tested alone. These relationships can be expressed mathematically by summing the fractional MIC values for all the components present in the mixture to give the “fractional inhibitory index”:

$$\sum FIC = {FIC}_{( component 1 )} + {FIC}_{( component 2 )}$$

Such that:
ΣFIC greater than 0.5 corresponds to indifferent, additive or antagonistic activity
ΣFIC less than or equal to 0.5 corresponds to synergistic activity

A comparable method is the calculation of the synergy index (SI) which is an industrial accepted method described by Kull, F.C.; Eisman, P.C.; Sylwestrowicz, H. D. and Mayer, R.L., in Applied Microbiology 9:538-541 (1961).

### Test Method

Liquid broth assays (MIC and checkerboard) were conducted to identify the minimum concentration(s) of individual and binary combinations of natural preservation materials. A modified methodology to ISO 20776-1:2006 was utilized for the screening as follows. Stock solutions of each preservation chemical at 200 ul/cell (or well) each and 100 ul of broth were created in separate 96-well plates. Each of the two-preservation chemical/broth mixes was then combined in a third 96 well plate at varying concentrations and then inoculated with 1-5x106 microorganisms and incubated at 30° C. for 24 hours, after which optical densities at OD600 nm were measured. MIC was defined as the concentration at which <25% growth was observed in comparison to a positive growth control containing no preservation chemicals.

Microorganism pools
Microorganism pools

| | |
|---|---|
| Pool 1 | Aspergillus brasiliensis |
| Pool 2 | Gram negative fermenting bacteria: |
| | Enterobacter gergoviae |

### Synergistic Combinations

Enterobacter gergoviae

### Harmol and Syringic Acid

Syringic Acid by itself displayed a minimal inhibitory concentration (MIC) of >50 mg/ml against Enterobacter gergoviae. Harmol by itself displayed a MIC of 0.625 mg/ml. In combination, 0.156 mg/ml Harmol and 3.125 mg/ml Syringic acid was needed to achieve the inhibitory effect. This illustrates the significant synergistic effect of harmol and syringic acid.

Aspergillus brasiliensis

### Harmol/Syringic Acid

Syringic Acid by itself displayed an MIC of >50 mg/ml against Aspergillus brasiliensis. Harmol by itself displayed a MIC of 12.5 mg/ml against Aspergillus brasiliensis. In combination, 1.563 mg/ml Harmol and 1.563 mg/ml Syringic acid was needed to achieve the inhibitory effect. This illustrates the significant synergistic effect of harmol and syringic acid.

The data provided in the above surprisingly demonstrates that when combining natural antimicrobial ingredients consistent with the present invention, a synergistic benefit against Enterobacter gergoviae and Aspergillus brasiliensis is observed.

1. An antimicrobial system comprising:
(a) a first component comprising a beta carboline alkaloid; and
(b) a second component comprising a phenolic acid, a phenolic acid salt or both,
wherein the beta carboline alkaloid is represented by the formula: 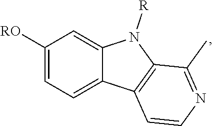
wherein each R is independently hydrogen or a C₁-C₄ alkyl, and the phenolic acid is represented by the formula: 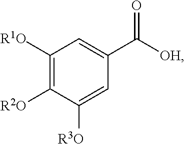
and further wherein each of R¹, R² and R³ is independently hydrogen or a C₁-C₄ alkyl,
the beta carboline alkaloid comprises 1-Methyl-9H-pyrido[3,4-b]indol-7-ol, the phenolic acid comprises 4-hydroxy-3,5-dimethoxybenzoic acid and the phenolic acid salt comprises a salt of 4-Hydroxy-3,5-dimethoxybenzoic acid.

2. The antimicrobial system according to claim 1, wherein the antimicrobial system comprises a sodium, potassium, calcium, magnesium and/or ammonium salt of the phenolic acid.

3. The antimicrobial system according to claim 1 wherein carboline alkaloid to phenolic acid or salt thereof are at a weight ratio from 0.75: 8 to 0.75:48.

4. An end use composition comprising an antimicrobial system comprising:
(a) a first component comprising a beta carboline alkaloid; and
(b) a second component comprising a phenolic acid, a phenolic acid salt or both; and
(c) a cosmetically acceptable carrier,
wherein the beta carboline alkaloid is represented by the formula: 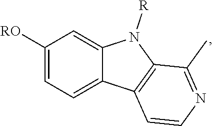
and each R is independently hydrogen or a C₁-C₄ alkyl, and the phenolic acid is represented by the formula: 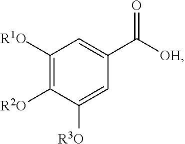
and each of R¹, R² and R³ is independently hydrogen or a C₁-C₄ alkyl, wherein the beta carboline alkaloid comprises 1-Methyl-9H-pyrido[3,4-b]indol-7-ol, the phenolic acid comprises 4-hydroxy-3,5-dimethoxybenzoic acid and the phenolic acid salt comprises a salt of 4-Hydroxy-3,5-dimethoxybenzoic acid.

5. The end use composition according to claim 4, wherein the composition comprises a sodium, potassium, calcium, magnesium and/or ammonium salt of the phenolic acid.

6. The end use composition according to claim 4, wherein the carboline alkaloid to phenolic acid or salt thereof are at a weight ratio from 0.75:8 to 0.75: 48.

7. The end use composition according to claim 4, wherein the composition is a shampoo, creams, lotions, serums, gels, balms, deodorants, antiperspirant, oral care composition, conditioners, bars and liquid wash product.

8. The end use composition according to claim 4, wherein the composition is substantially free of paraben, formaldehyde donor, phenoxyethanol, silicone, sulfate or mixtures thereof and further comprises terpineol, thymol, niacinamide, 12-hydroxystearic acid, a retinol, a resorcinol, or a mixture thereof.

9. The end use composition according to claim 4, wherein the antimicrobial system preserves the end use composition and is an antimicrobial agent to a surface it contacts.

10. The end use composition according to claim 4, wherein the antimicrobial system comprises from 0.7 to 5% by weight carboline alkaloid based on total weight of carboline alkaloid and phenolic acid or salt thereof in the antimicrobial system, or the antimicrobial system comprises from 45 to 78% by weight carboline alkaloid based on total weight of carboline alkaloid and phenolic acid or salts thereof in the antimicrobial system.

11. The end use composition according to claim 4 wherein the composition further comprises niacinamide, biotin, vitamin C, vitamin B₂, folic acid, vitamin B₆, or a mixture thereof.

12. The end use composition according to claim 4 wherein the composition further comprises hydroxypropyl starch phosphate.

13. The end use composition according to claim 4 wherein the composition further comprises avocado oil, coconut oil, shea butter, 12-hydroxystearic acid retinol propionate or a mixture thereof.
